(19) United States
(12) Patent Application Publication
(10) Pub. No.: US 2023/0303493 A1
(43) Pub. Date: Sep. 28, 2023

(54) ANTIBACTERIAL ADJUVANTS AND APPLICATIONS THEREOF

(71) Applicants: THE TRUSTEES OF PRINCETON UNIVERSITY, Princeton, NJ (US); SAN JOSE STATE UNIVERSITY RESEARCH FOUNDATION, San Jose, CA (US)

(72) Inventors: Laura Miller Conrad, Redwood City, CA (US); Martin F. Semmelhack, Princeton, NJ (US); Zinan Zhang, Decatur, GA (US); Minh Tran, San Francisco, CA (US); Lauren Blankenship, College Station, TX (US)

(21) Appl. No.: 18/021,284

(22) PCT Filed: Aug. 13, 2021
(86) PCT No.: PCT/US2021/045929

§ 371 Date: Feb. 14, 2023

Related U.S. Application Data
(60) Provisional application No. 63/065,701, filed on Aug. 14, 2020.

(51) Int. Cl. C07D 213/75; A61K 38/12; A61P 31/04

(52) U.S. Cl. CPC C07D 213/75; A61K 38/12; A61P 31/04

(57) ABSTRACT

In one aspect, compounds and methods are described herein for treating gram negative bacteria having resistance to cationic antimicrobial peptides. In some embodiments, for example, compounds of formula I are provided. In another aspect, a treatment for a gram negative bacterial infection comprises a cationic antimicrobial protein (CAP) in conjunction with a compound of formula I.

### RELATED APPLICATION DATA

The present application claims priority pursuant to Section 8 of the Patent Cooperation Treaty to U.S. Provisional Patent Application Ser. No. 63/065,701 filed Aug. 14, 2020 which is incorporated herein by reference in its entirety.

### STATEMENT OF GOVERNMENT RIGHTS

This invention was made with government support under Grant No. GM118199 awarded by the National Institutes of Health and support under Grant No. DUE-1258366 awarded by the National Science Foundation. The government has certain rights in the invention.

### FIELD

The present invention relates to small molecule antibacterial adjuvants and, in particular, to compounds increasing the susceptibility of gram-negative bacteria to cationic antimicrobial peptides.

### BACKGROUND

The development of antibiotics was one of the great discoveries of the 20^{th} century. However, the rise in antibiotic-resistant strains is hampering our ability to continue to successfully treat bacterial infections. Within the United States alone, the Centers for Disease Control and Prevention (CDC) made a conservative estimate in 2013 that over two million individuals are infected with antibiotic-resistant strains each year, with 23,000 succumbing to the infection. The World Health Organization (WHO) also found high levels of resistant bacteria worldwide. While methicillin-resistant Staphylococcus aureus (MRSA) has occupied much of the headlines, gram-negative bacteria are also an increasing threat. For example, in February and March of 2015 there were 2 outbreaks of carbapenem-resistant Enterobacteriaceae (CRE) from contaminated duodenoscopes at UCLA Medical Center and Cedars-Sinai. The situation will only continue to worsen without new strategies to combat bacterial infections.

Multidrug-resistant strains of Pseudomonas aeruginosa have been identified as a serious threat by the CDC. P. aeruginosa is a versatile gram-negative pathogen that is difficult to eradicate from hospital settings where it causes a variety of secondary infections and is especially prevalent in patients with burn wounds and in those with implanted medical devices. Cystic fibrosis (CF) patients are an especially sensitive population. Most CF patients will acquire a P. aeruginosa infection, which is the leading cause of mortality for CF patients. Few treatments are available for P. aeruginosa, and drug-resistant strains are increasingly encountered. Cationic antimicrobial peptides (CAPs) like colistin are currently used as last resort drugs for multidrug resistant strains and are being used in patients with cystic fibrosis and with severe burns with increasing frequency. Alarmingly, CAP-resistant strains have been identified placing additional pressure on the need for treatment options for increasingly resistant strains of bacteria.

### SUMMARY

In one aspect, compounds and methods are described herein for treating gram negative bacteria having resistance to cationic antimicrobial peptides. In some embodiments, for example, compounds of formula I are provided:

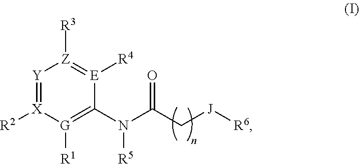

wherein E, G, X, Y, and Z are independently selected from C and N; and
wherein J is selected from the group consisting of O, S, CH₂, and NR⁷, wherein R⁷ is selected from the group consisting of hydrogen and alkyl; and
wherein R¹-R⁴ are independently selected from the group consisting of hydrogen, alkyl, fluoroalkyl, halo, and —OR⁸ wherein R⁸ is selected from the group consisting of hydrogen, alkyl and alkenyl; and
wherein R⁵ is selected from the group consisting of hydrogen and alkyl; and
wherein R⁶ is selected from the group consisting of hydrogen, alkenyl, alkynl, cycloalkyl, heterocycloalkyl, fused aryl, heteroaryl, fused heteroaryl, -arylene-alkynyl, -heteroarylene-alkynl, -arylene-aryl, -arylene-heteroaryl, -aryelene-thioalkyl, -arylene-(t-butyl), -heteroarylene-(t-butyl), -aryelene-amine, -heteroarylene-amine, -arylene-C(O)R⁹ wherein R⁹ is selected from the group consisting of hydrogen, alkyl, alkoxy, amine, and —OBn; and
wherein the cycloalkyl and heterocycloalkyl are optionally substituted with one or more substituents selected from the group consisting of alkyl, aryl, amine, heteroaryl, halo, hydroxy, and alkoxy; and
wherein n is an integer from 1 to 10.

In another aspect, a treatment for a gram negative bacterial infection comprises a cationic antimicrobial protein (CAP) in conjunction with a compound of formula I:

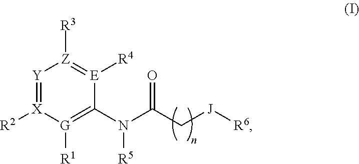

wherein E, G, X, Y, and Z are independently selected from C and N; and
wherein J is selected from the group consisting of O, S, CH₂, and NR⁷, wherein R⁷ is selected from the group consisting of hydrogen and alkyl; and
wherein R¹-R⁴ are independently selected from the group consisting of hydrogen, alkyl, fluoroalkyl, halo, and —OR⁸ wherein R⁸ is selected from the group consisting of hydrogen, alkyl and alkenyl; and
wherein R⁵ is selected from the group consisting of hydrogen and alkyl; and
wherein R⁶ is selected from the group consisting of hydrogen, alkenyl, alkynl, cycloalkyl, heterocycloalkyl, fused aryl, heteroaryl, fused heteroaryl, -arylene-alkynyl, -heteroarylene-alkynl, -arylene-aryl, -arylene-heteroaryl, -aryelene-thioalkyl, -arylene-(t-butyl), -heteroarylene-(t-butyl), -aryelene-amine, -heteroarylene-amine, -arylene-C(O)R⁹ wherein R⁹ is selected from the group consisting of hydrogen, alkyl, alkoxy, amine, and —OBn; and
wherein the cycloalkyl and heterocycloalkyl are optionally substituted with one or more substituents selected from the group consisting of alkyl, aryl, amine, heteroaryl, halo, hydroxy, and alkoxy; and
wherein n is an integer from 1 to 10.

In another aspect, methods of treating bacterial infections are described herein. In some embodiments, a method of inhibiting growth of gram negative bacteria comprises treating the gram negative bacteria with a cationic antimicrobial peptide (CAP) in conjunction with a compound of formula I:

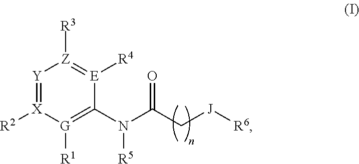

wherein E, G, X, Y, and Z are independently selected from C and N; and
wherein J is selected from the group consisting of O, S, CH₂, and NR⁷, wherein R⁷ is selected from the group consisting of hydrogen and alkyl; and
wherein R¹-R⁴ are independently selected from the group consisting of hydrogen, alkyl, fluoroalkyl, halo, and —OR⁸ wherein R⁸ is selected from the group consisting of hydrogen, alkyl and alkenyl; and
wherein R⁵ is selected from the group consisting of hydrogen and alkyl; and
wherein R⁶ is selected from the group consisting of hydrogen, alkenyl, alkynl, cycloalkyl, heterocycloalkyl, fused aryl, heteroaryl, fused heteroaryl, -arylene-alkynyl, -heteroarylene-alkynl, -arylene-aryl, -arylene-heteroaryl, -aryelene-thioalkyl, -arylene-(t-butyl), -heteroarylene-(t-butyl), -aryelene-amine, -heteroarylene-amine, -arylene-C(O)R⁹ wherein R⁹ is selected from the group consisting of hydrogen, alkyl, alkoxy, amine, and —OBn; and
wherein the cycloalkyl and heterocycloalkyl are optionally substituted with one or more substituents selected from the group consisting of alkyl, aryl, amine, heteroaryl, halo, hydroxy, and alkoxy; and
wherein n is an integer from 1 to 10.

In some embodiments, compounds of formula I can inhibit one or more CAP resistant mechanisms of the gram negative bacteria including, but not limited to, structural modifications to the outer membrane surface of the gram negative bacteria. Compounds of formula I, for example, can inhibit alteration of the charge of the outer membrane surface. In some embodiments, compounds of formula I can inhibit structural changes that reduce or mask negative charge of the outer membrane surface, thereby maintaining ionic interaction between the CAP and outer membrane surface. Additionally, compounds of formula I can reduce the minimum inhibitory concentration of the CAP when treating gram negative bacteria. In some embodiments, compounds of formula I are used in conjunction with a CAP to treat P. aeruginosa.

These and other embodiments are further described in the following detail description.

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “alkynyl” as used herein, alone or in combination, refers to a straight or branched chain hydrocarbon group having at least one carbon-carbon triple bond and optionally substituted with one or more substituents.

The term “aryl” as used herein, alone or in combination, refers to an aromatic monocyclic or multicyclic ring system optionally substituted with one or more ring substituents.

The term “heteroaryl” as used herein, alone or in combination, refers to an aromatic monocyclic or multicyclic ring system in which one or more of the ring atoms is an element other than carbon, such as nitrogen, boron, oxygen and/or sulfur.

The term “cycloalkyl” as used herein, alone or in combination, refers to a non-aromatic, mono- or multicyclic ring system optionally substituted with one or more ring substituents.

The term “heterocycloalkyl” as used herein, alone or in combination, refers to a non-aromatic, mono- or multicyclic ring system in which one or more of the atoms in the ring system is an element other than carbon, such as boron, nitrogen, oxygen, sulfur or phosphorus, alone or in combination, and wherein the ring system is optionally substituted with one or more ring substituents.

The term “alkoxy” as used herein, alone or in combination, refers to the moiety —OR, where R is alkyl, alkenyl, or aryl defined above.

The term “halo” as used herein, alone or in combination, refers to elements of Group VIIA of the Periodic Table (halogens). Depending on chemical environment, halo can be in a neutral or anionic state.

### I. Compounds and Pharmaceutical Compositions for Treating Bacterial Infections

Various compounds are described herein. As discussed above and further illustrated in the examples below, compounds falling under formula I can exhibit antibacterial properties in conjunction with one or more CAPs, in some embodiments. Pharmaceutical compositions employing such compounds are also provided. Compounds for formula I can be individually administered in any amount consistent with treating bacterial infections. In some embodiments, a treatment for a gram negative bacterial infection comprises a cationic antimicrobial protein (CAP) in conjunction with a compound of formula I. The CAP and compound of formula I can be administered as a single composition or as separate compositions. In some embodiments, a compound of formula I exhibits an IC₅₀<20 μM for inhibiting growth of one or more bacterial species in the presence of the CAP. For example, a compound of formula I, in some embodiments, can exhibit an IC₅₀ selected from Table I for inhibiting P. aeruginosa growth in conjunction with the administration of colistin. Colistin can be administered in the rage of 0.007 μg/mL to 16 μg/mL, such as 0.25 to 0.5 μg/mL in some embodiments.

TABLE I
IC₅₀ of Compound of formula I (μM)
≤15
≤10
≤5
0.1-10
0.5-5

The amount or concentration of compounds of formula I employed in conjunction with CAPs can be dependent on the identity and/or nature of the bacteria being treated, including gram negative bacteria. In some embodiments, two or more differing compounds falling under formula I can be combined with CAP for the treatment of bacterial infections.

### II. Methods of Treating Bacterial Infections

In some embodiments, a method of inhibiting growth of gram negative bacteria comprises treating the gram negative bacteria with a cationic antimicrobial peptide (CAP) in conjunction with a compound of formula I. In some embodiments, two or more differing compounds falling under formula I can be combined with CAP for the treatment of bacterial infections.

These and other embodiments are further illustrated in the following non-limiting examples.

### EXAMPLES—ANTIBACTERIAL ADJUVANT COMPOUNDS

Non-limiting examples of compounds of formula I and other compounds increasing gram negative bacteria susceptibility to CAPs are synthesized according to the following procedures. General chemical methods. Unless otherwise stated, reactions were performed in flame-dried glassware fitted with rubber septa under nitrogen atmosphere and were stirred with Teflon-coated magnetic stirring bars. Liquid reagents and solvents were transferred via syringe using standard Schlenk techniques. Reaction solvents were dried by passage over a column of activated alumina if noted. All other solvents and reagents were used as received unless otherwise noted. Reaction temperatures above 23° C. refer to oil bath temperature, which was controlled by an OptiCHEM or IKA RCT Basic temperature modulator. Thin layer chromatography was performed using SiliCycle silica gel 60 F-254 precoated plates (0.25 mm) and visualized by UV irradiation and anisaldehyde or potassium permanganate stain. Sorbent standard silica gel (particle size 40-63 μm) or SiliCycle Silica-P silica gel (particle size 40-63 μm) was used for flash chromatography. ¹H and ¹³C NMR spectra were recorded on a Bruker Avance III (500 MHz for ¹H; 125 MHz for ¹³C) spectrometer fitted with either a ¹H-optimized TCI (H/C/N) cryoprobe or a ¹³C-optimized dual C/H cryoprobe, a Bruker NanoBay (300 MHz for ¹H, 75 MHz for ¹³C), or a Varian Inova (400 MHz for ¹H; 100 MHz for ¹³C) spectrometer. Chemical shifts (δ) are reported in ppm relative to the residual solvent signal (δ=7.26 for 1H NMR and δ=77.0 for 13C NMR for CDCl₃). Data for 1H NMR spectra are reported as follows: chemical shift (multiplicity, coupling constants, number of hydrogens). Abbreviations are as follows: s (singlet), d (doublet), t (triplet), q (quartet), p (pentet), dd (doublet of doublets), ddd (doublet of doublet of doublets), dt (doublet of triplets), ddt (doublet of doublet of triplets), td (triplet of doublets), tt (triplet of triplets), m (multiplet). High-resolution mass spectral analysis was performed using an Agilent 1200-series electrospray ionization—time-of-flight (ESI-TOF) mass spectrometer in the positive ESI mode.

### Synthesis of Analog 3

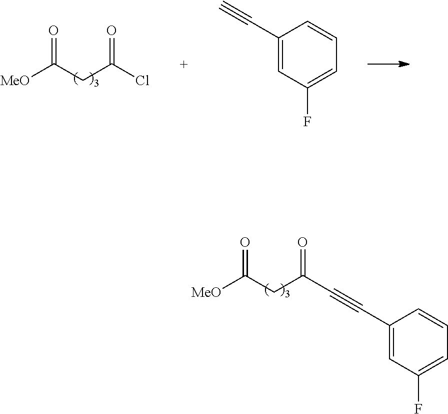

Methyl 7-(3-fluorophenyl)-5-oxohept-6-ynoate (8). Hexamethyldisilazane (1.32 mL, 6.05 mmol) was dissolved in anhydrous tetrahydrofuran (dried over a column of activated alumina,10 mL) and stirred under nitrogen. The solution was cooled to −78° C. and 2.5 M n-butyllithium in hexanes (2.42 mL, 6.05 mmol) was added dropwise. The reaction mixture was stirred for 5 min. at −78 ° C. and 20 min. at room temperature. The reaction mixture was then recooled to −78 ° C., and 3-fluorophenylacetylene (0.64 mL, 5.50 mmol) dissolved in anhydrous tetrahydrofuran (dried over a column of activated alumina,1 mL) was added. In another round bottom flask, glutaric acid monomethyl ester chloride (1.52 mL, 11.0 mmol) was dissolved in anhydrous tetrahydrofuran (dried over a column of activated alumina,10 mL) and cooled to −78 ° C. The acid chloride solution was cannulated to the lithiated alkyne reaction mixture. Post-cannulation, the reaction mixture was stirred for 30 min. at −78° C. and then 1 h at room temperature. The reaction was quenched using saturated NaHCO₃ (20 mL). The organic layer was extracted using ethyl acetate (3×25 mL), washed with water (2×20 mL) and brine (30 mL), dried over Na2SO4 and concentrated. The crude product was purified using flash chromatography to obtain the product in 31% yield. ¹H NMR (500 MHz, CDCl₃) δ7.41-7.31 (m, 2H), 7.27 (d, J=3.9 Hz, 1H), 7.17 (ddt, J=8.4, 5.0, 2.6 Hz, 1H), 3.69 (d, J=1.0 Hz, 3H), 2.77 (t, J=7.2 Hz, 2H), 2.42 (t, J=7.3 Hz, 2H), 2.10-1.99 (m, 2H); ¹³C NMR (125 MHz, CDCl₃) δ186.6, 173.3, 162.5, 130.4, 128.9, 121.6, 119.8, 118.1, 89.0, 87.8, 51.7, 44.4, 32.8, 19.0; HRMS (ESI-TOF) calculated for C₁₄H₁₃FO₃ [M+H]⁺: m/z 249.0927, found 249.0913.

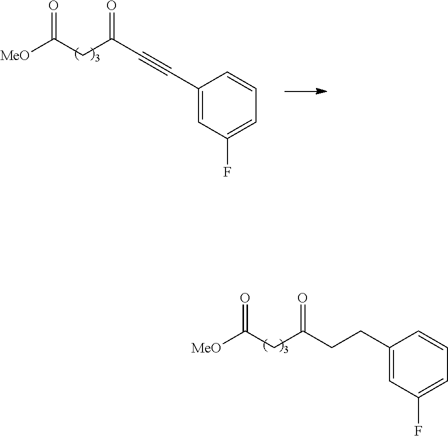

Methyl 7-(3-fluorophenyl)-5-oxoheptanoate (S3). Ynoate 8 (0.430 g, 1.70 mmol) was dissolved in methanol (30 mL). Palladium on charcoal (0.080 g, 10 wt%) was added to the solution. The reaction mixture was hydrogenated at room temperature and atmospheric pressure overnight. Then the reaction mixture was filtered through celite and concentrated. No further purification was necessary to obtain the product in a 99% yield. ¹H NMR (500 MHz, CDCl3) δ7.23 (ddd, J=9.2, 7.8, 6.1 Hz, 1H), 6.95 (d, J=7.6 Hz, 1H), 6.92-6.82 (m, 2H), 3.66 (s, 3H), 2.89 (t, J=7.6 Hz, 2H), 2.72 (t, J=7.5 Hz, 2H), 2.46 (t, J=7.2 Hz, 2H), 2.32 (t, J=7.2 Hz, 2H), 1.89 (p, J=7.2 Hz, 2H); ¹³C NMR (125 MHz, CDCl₃) δ208.7, 173.6, 162.9, 143.5, 130.0, 124.0, 115.3, 112.9, 51.6, 43.8, 41.7, 33.0, 29.3, 18.8; HRMS (ESI-TOF) calculated for C₁₄H₁₇FO₃ [M+Na]⁺: m/z 275.1059, found 275.1045.

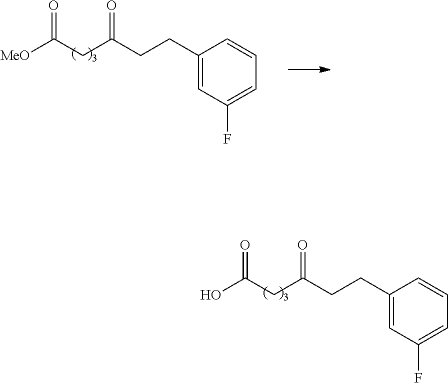

7-(3-Fluorophenyl)-5-oxoheptanoic acid (9). Methyl ester S3 (0.429 g, 1.70 mmol) was dissolved in methanol (10 mL), and 1 M NaOH (3 mL) was added to the solution. The reaction mixture was stirred overnight at room temperature. The methanol was evaporated, and the aqueous layer was diluted with water and washed with ethyl acetate (2×15 mL). The aqueous layer was then acidified with 1 M HCl to pH 2. The pure product was extracted with ethyl acetate (3×15 mL), dried over Na₂SO₄ and concentrated. No further purification was necessary to obtain the product in an 87% yield. ¹H NMR (500 MHz, CDCl₃) δ7.26-7.17 (m, 1H), 6.95 (d, J=7.6 Hz, 1H), 6.88 (tt, J=7.2, 2.1 Hz, 2H), 2.89 (t, J=7.5 Hz, 2H), 2.73 (t, J=7.5 Hz, 2H), 2.49 (t, J=7.2 Hz, 2H), 2.37 (t, J=7.2 Hz, 2H), 1.90 (q, J=7.2 Hz, 2H); ¹³C NMR (125 MHz, CDC₁₃) δ208.7, 179.0, 162.9, 143.5, 129.9, 123.9, 115.1, 112.9, 43.9, 41.5, 32.8, 29.3, 18.4; HRMS (ESI-TOF) calculated for C₁₃H₁₅FO₃ [M+H]⁺: m/z 261.0903, found 261.0889.

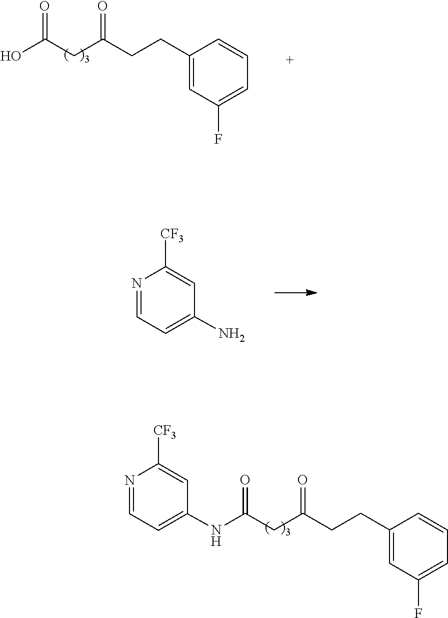

7-(3-Fluorophenyl)-5-oxo-N-(2-(trifluoromethyl)pyridin-4-yl)heptanamide (3). Carboxylic acid 9 (0.020 g, 0.084 mmol), 4-amino-2-(trifluoromethyl)pyridine (0.014 g, 0,084 mmol), and 1-methyl-2-chloropyridinium iodide (0.026 g, 0.10 mmol) were added to a stirring solution of triethylamine (0.03 mL) and anhydrous dichloromethane (dried over a column of activated alumina, 1 mL) in a 10 mL round bottom flask. The reaction was stirred at room temperature for 48 h. The solution was poured onto distilled water (5 mL). The organic layer was extracted with dichloromethane (3×5 mL). The combined organic layers were then dried over Na₂SO₄, filtered, and evaporated. The crude product was purified using flash chromatography to furnish the compound 3 in a 19% yield. ¹H NMR (500 MHz, CDCl₃) δ8.59 (d, J=5.6 Hz, 1H), 8.33 (s, 1H), 7.98 (d, J=2.2 Hz, 1H), 7.74 (dd, J=5.8, 2.2 Hz, 1H), 7.25-7.16 (m, 1H), 6.95 (d, J=7.7 Hz, 1H), 6.88 (ddd, J=9.4, 3.9, 1.8 Hz, 2H), 2.90 (q, J=6.8, 6.0 Hz, 2H), 2.78 (t, J=7.4 Hz, 2H), 2.56 (t, J=6.6 Hz, 2H), 2.42 (t, J=7.1 Hz, 2H), 1.98 (p, J=6.9 Hz, 2H); ¹³C NMR (125 MHz, CDCl₃) δ210.0, 171.8, 162.9, 150.4, 146.6, 130.0, 124.0, 121.4, 115.4, 115.3, 115.1, 113.2, 113.1, 110.5, 43.9, 41.4, 36.4, 29.3, 19.0; HRMS (ESI-TOF) calculated for C₁₉H₁₈F₄N₂O₂ [M+H]⁺: m/z 383.1383, found 383.1372.

### General Procedure A

The respective 4-aminopyridine (1 equiv), CH₂Cl₂ (0.15 M), and pyridine (2 equiv) were combined in a flame-dried flask. The reaction mixture was cooled to 0° C., and the acid chloride (1 equiv) was added dropwise. The reaction mixture was allowed to warm to room temperature over 3-5 h. The reaction was then quenched with saturated aqueous NaHCO₃. The layers were separated, and the aqueous layer was extracted 3× with CH₂Cl₂ . The combined organic layer was washed sequentially with 1 M HCl and brine, then dried over MgSO₄ and concentrated. The crude product was purified by column chromatography.

### General Procedure B

The alkyl halide (1 equiv), anhydrous potassium iodide (12 equiv), anhydrous potassium carbonate (7.5 equiv), and the aryl nucleophile (3.8 equiv) were dissolved in DMF (0.68 M). The reaction was stirred for 3-5 days at room temperature until complete by TLC. The reaction was quenched with water and extracted with CH₂Cl₂ . The combined organic layer was washed sequentially with saturated aqueous NaHCO3 (2×), 1 M HCl and brine. The solution was dried over MgSO₄ and concentrated. The crude product was purified by column chromatography.

### General Procedure C

The ester (1.0 equiv) was added to a solution of lithium hydroxide monohydrate (5.0 equiv) in 3:1 THF/H₂O (0.30 M). The reaction was heated to 65° C. for 12 h, or until complete by TLC. The reaction was cooled and acidified with 1 M HCl. The aqueous layer was extracted 3× with EtOAc. The combined organic layer was washed with brine, dried over Na₂SO₄, filtered and concentrated. The product was carried forward crude.

### General Procedure D

To a flame-dried flask were added the carboxylic acid (1.0 equiv), dicyclohexylcarbodiimide (1.1 equiv), dimethylaminopyridine (1.1 equiv), the amine (1.0 equiv), and CH₂Cl₂ (0.40 M). After stirring at room temperature for 24 h, the reaction mixture was filtered through a Celite plug and concentrated. The crude product was purified by column chromatography.

### General Procedure E

To solution containing palladium(II) triphenylphosphine (0.05 equiv), triphenylphosphine (0.025 equiv) in THF (0.1 M), a bromophenol (1 equiv), triethylamine (1.5 equiv) and trimethylsilyl-acetylene (1.5 equiv) were added. The reaction was stirred for 20 min at room temperature before adding copper iodide (0.02 equiv). The reaction was stirred overnight, then filtered through Celite, concentrated and purified by column chromatography.

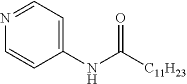

N-(pyridin-4-yl)dodecanamide (S1). Prepared from 4-aminopyridine and lauroyl chloride using general procedure A to furnish S1 in a 50% yield.

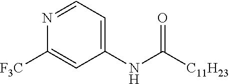

N-(2-(trifluoromethyl)pyridin-4-yl)dodecanamide (S2). Prepared from 4-amino-2-(trifluoromethyl)pyridine and lauroyl chloride using general procedure A to furnish S2 in a 90% yield.

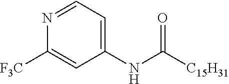

20 N-(2-(trifluoromethyl)pyridin-4-yl)palmitamide (S4). Prepared from 4-amino-2-(trifluoromethyl)pyridine and palmitoyl chloride using general procedure A to furnish S4 in a 57% yield.

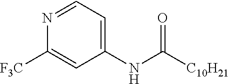

N-(2-(trifluoromethyl)pyridin-4-yl)undecanamide (S5). Prepared from 4-amino-2-(trifluoromethyl)pyridine and undecanoyl chloride using general procedure A to produce S5 in a 72% yield.

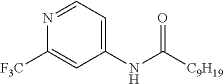

N-(2-(trifluoromethyl)pyridin-4-yl)decanamide (S6). Prepared from 4-amino-2-(trifluoromethyl)pyridine and decanoyl chloride using general procedure A to furnish S6 in a 74% yield.

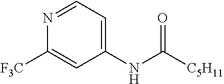

N-(2-(trifluoromethyl)pyridin-4-yl)hexanamide (S7). Prepared from 4-amino-2-(trifluoromethyl)pyridine and hexanoyl chloride using general procedure A to give S7 in a 64% yield.

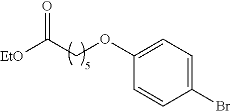

Ethyl 6-(4-bromophenoxy)hexanoate (S8). Prepared from ethyl 6-bromohexanoate andp-bromophenol using general procedure B to obtain S8 in a 65% yield.

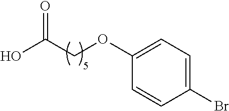

6-(4-Bromophenoxy)hexanoic acid (S9). Prepared from S8 using general procedure C to give S9 in a 28% yield.

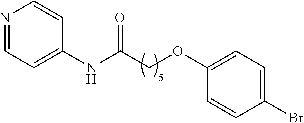

6-(4-Bromophenoxy)-N-(pyridin-4-yl)hexanamide (S10). Prepared from S9 and 4-aminopyridine using general procedure D to obtain S10 in a 48% yield.

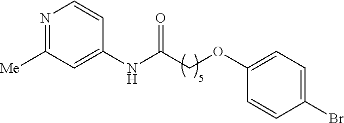

6-(4-Bromophenoxy)-N-(2-methylpyridin-4-yl)hexanamide (S11). Prepared from S9 and 4-amino-2-methylpyridine using general procedure D to provide S11 in an 84% yield.

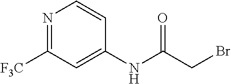 [SMILES: O=C(CBr)Nc1ccnc(C(F)(F)F)c1]

6-Bromo-N-(2-(trifluoromethyl)pyridin-4-yl)-hexanamide (S12). Prepared from 4-amino-2-10 (trifluoromethyl)pyridine and 6-bromohexanoyl chloride using general procedure A to furnish S12 in a 90% yield.

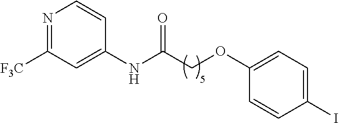

6-(4-Iodophenoxy)-N-(2-(trifluoromethyl)pyridin-4-yl)hexanamide (S13). Prepared from amide S12 and p-iodophenol using general procedure B to furnish S13 in a 45% yield.

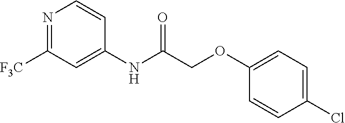 [SMILES: O=C(COc1ccc(Cl)cc1)Nc1ccnc(C(F)(F)F)c1]

6-(4-Chlorophenoxy)-N-(2-(trifluoromethyl)pyridin-4-yl)hexanamide (S14). Prepared from amide S12 and p-chlorophenol using general procedure B to provide S14 in a 34% yield.

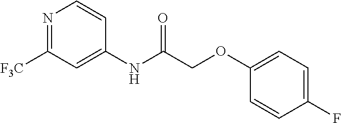 [SMILES: O=C(COc1ccc(F)cc1)Nc1ccnc(C(F)(F)F)c1]

6-(4-Fluorophenoxy)-N-(2-(trifluoromethyl)pyridin-4-yl)hexanamide (S15). Prepared from amide S12 and 4-fluorophenol using general procedure B to furnish S15 in a 37% yield.

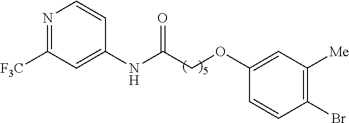

6-(4-Bromo-3-methylphenoxy)-N-(2-(trifluoromethyl)pyridin-4-yl)hexanamide (S16). Prepared from amide S12 and 3-methyl-4-bromophenol using general procedure B to give S16 in a 23% yield.

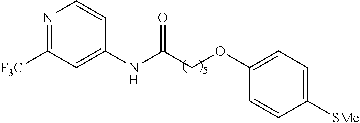

6-(4-(Methylthio)phenoxy)-N-(2-(trifluoromethyl)pyridin-4-yl)hexanamide (S17). Prepared from amide S12 and 4-(methylthio)phenol using general procedure B to provide S17 in a 57% yield.

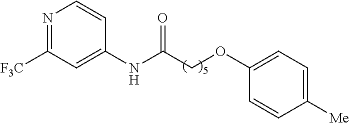

6-(p-Tolyloxy)-N-(2-(trifluoromethyl)pyridin-4-yl)hexanamide (S18). Prepared from amide S12 and 4-methylphenol using general procedure B to give S18 in a 51% yield.

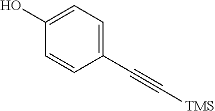 [SMILES: C[Si](C)(C)C#Cc1ccc(O)cc1]

4-((Trimethylsilyl)ethynyl)phenol (S19). Prepared from p-bromophenol using general procedure E to furnish S19 in a quantitative yield.

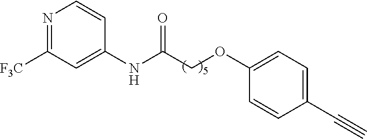

6-(4-Ethynylphenoxy)-N-(2-(trifluoromethyl)pyridin-4-yl)hexanamide (S20). Prepared from amide S12 and S19 using general procedure B to furnish S20 in a 16% yield.

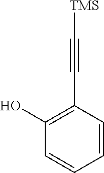 [SMILES: C[Si](C)(C)C#Cc1ccccc1O]

2-((Trimethylsilyl)ethynyl)phenol (S21). Prepared from o-bromophenol using general procedure E to produce S21 in a quantitative yield.

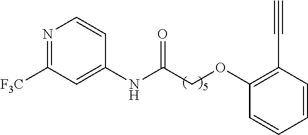

6-(2-Ethynylphenoxy)-N-(2-(trifluoromethyl)pyridin-4-yl)hexanamide (S22). Prepared from amide S12 and S21 using general procedure B to obtain S22 in a 10% yield.

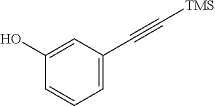 [SMILES: C[Si](C)(C)C#Cc1cccc(O)c1]

2-((Trimethylsilyl)ethynyl)phenol (S23). Prepared from m-bromophenol using general procedure E to give S23 in a quantitative yield.

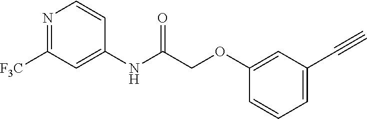 [SMILES: C#Cc1cccc(OCC(=O)Nc2ccnc(C(F)(F)F)c2)c1]

6-(3-Ethynylphenoxy)-N-(2-(trifluoromethyl)pyridin-4-yl)hexanamide (S24). Prepared from amide S12 and S23 using general procedure B to give S24 in a 16% yield.

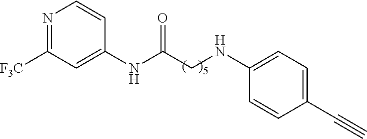

6-((4-Ethynylphenyl)amino)-N-(2-(trifluoromethyl)pyridin-4-yl)hexanamide (S25). Prepared from amide S12 and 4-ethynylailine using general procedure B to give S25 in a 63% yield.

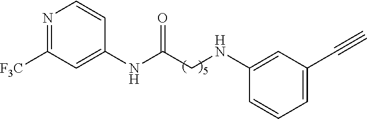

6-((2-Ethynylphenyl)amino)-N-(2-(trifluoromethyl)pyridin-4-yl)hexanamide (S26). Prepared from amide S12 and 3-ethynylaniline using general procedure B to furnish S26 in a 38% yield.

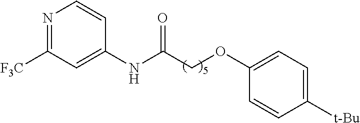

6-(4-(tert-Butyl)phenoxy)-N-(2-(trifluoromethyl)pyridin-4-yl)hexanamide (S27). Prepared from amide S12 and 4-t-butylphenol using general procedure B to furnish S27 in a 20% yield.

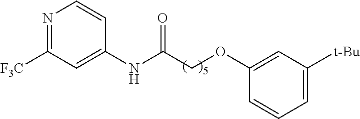

6-(3-(tert-Butyl)phenoxy)-N-(2-(trifluoromethyl)pyridin-4-yl)hexanamide (S28). Prepared from amide S12 and 3-t-butylphenol using general procedure B to furnish S28 in an 18% yield.

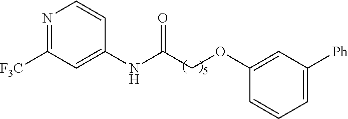

6-([1,r-Biphenyl]-3-yloxy)-N-(2-(trifluoromethyl)pyridin-4-yl)hexanamide (S29). Prepared from amide S12 and 3-phenylphenol using general procedure B to produce S29 in a 27% yield.

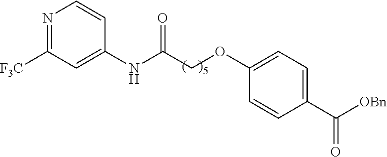

Benzyl 4-((6-oxo-6((2-(trifluoromethyl)pyridin-4-yl)amino)hexyl)oxy)benzoate (S30). Prepared from amide S12 and benzyl 4-hydroxybenzoate using general procedure B to furnish S30 in a 42% yield.

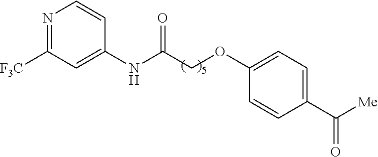

6-(4-Acetylphenoxy)-N-(2-(trifluoromethyl)pyridin-4-yl)hexanamide (S31). Prepared from amide S12 and 4-hydroxyacetophenone using general procedure B to furnish S31 in a 30% yield.

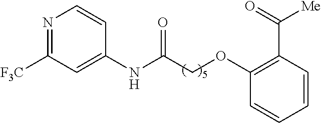

6-(2-Acetylphenoxy)-N-(2-(trifluoromethyl)pyridin-4-yl)hexanamide (S32). Prepared from amide S12 and 2-hydroxyacetophenone using general procedure B to obtain S32 in a 60% yield.

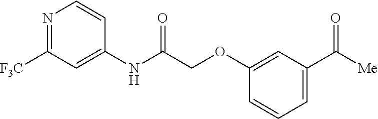 [SMILES: CC(=O)c1cccc(OCC(=O)Nc2ccnc(C(F)(F)F)c2)c1]

6-(3-Acetylphenoxy)-N-(2-(trifluoromethyl)pyridin-4-yl)hexanamide (S33). Prepared from amide S12 and 3-hydroxyacetophenone using general procedure B to produce S33 in an 8% yield.

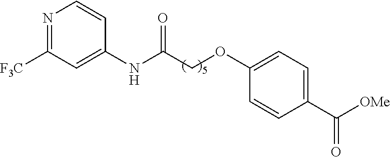

Methyl 4-((6-oxo-6-((2-(trifluoromethyl)pyridin-4-yl)amino)hexyl)oxy)benzoate (S34). Prepared from amide S12 and methyl-4-hydroxybenzoate using general procedure B to obtain S34 in a 46% yield.

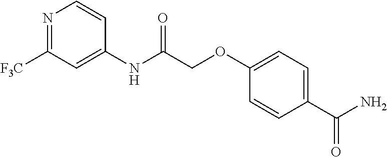 [SMILES: NC(=O)c1ccc(OCC(=O)Nc2ccnc(C(F)(F)F)c2)cc1]

4-((6-oxo-6-((2-(trifluoromethyl)pyridin-4-yl)amino)hexyl)oxy)benzamide (S35). Prepared from amide S12 and 4-hydroxybenzamide using general procedure B to produce S35 in a 20% yield.

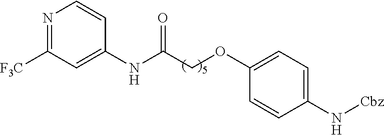

Benzyl (4-((6-oxo-6-((2-(trifluoromethyl)pyridin-4-yHamino)hexyl)oxy)phenyl)carbamate (S36). Prepared from amide S12 and benzyl (4-hydroxyphenyl)carbamate using general procedure B to give S36 in a 22% yield.

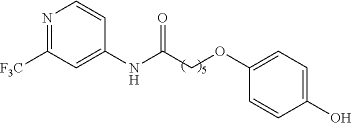

6-(4-Hydroxyphenoxy)-N-(2-(trifluoromethyl)pyridin-4-yl)hexanamide (S37). Prepared from amide S12 and hydroquinone using general procedure B to obtain S37 in a 2% yield.

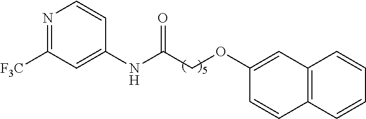

6-(Naphthalen-2-yloxy)-N-(2-(trifluoromethyl)pyridin-4-yl)hexanamide (S38). Prepared from amide S12 and 2-naphthol using general procedure B to give S38 in a 32% yield.

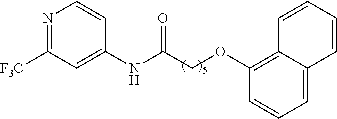

6-(Naphthalen-1-yloxy)-N-(2-(trifluoromethyl)pyridin-4-yl)hexanamide (S39). Prepared from amide S12 and 1-naphthol using general procedure B to furnish S39 in a 26% yield.

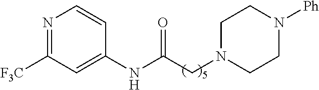

6-(4-Phenylpiperazin-1-yl)-N-(2-(trifluoromethyl)pyridin-4-yl)hexanamide (S40). Prepared from amide S12 and 1-phenylpiperazine using general procedure B to provide S40 in a 25% yield.

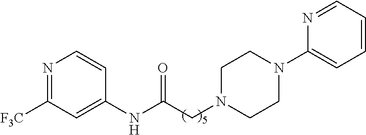

6-(4-(Pyridin-2-yl)piperazin-1-yl)-N-(2-(trifluoromethyl)pyridin-4-yl)hexanamide (S41). Prepared from amide S12 and 1-(2-pyridyl)piperazine using general procedure B to obtain S41 in a 22% yield.

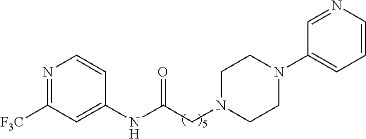

6-(4-(Pyridin-3-yl)piperazin-1-yl)-N-(2-(trifluoromethyl)pyridin-4-yl)hexanamide (S42). Prepared from amide S12 and 1-pyridin-3-ylpiperzine dihydrobromide using general procedure B to give S42 in a 30% yield.

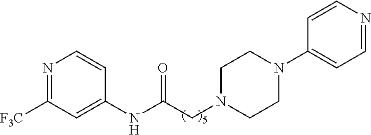

6-(4-(Pyridin-4-yl)piperazin-1-yl)-N-(2-(trifluoromethyl)pyridin-4-yl)hexanamide (S43). Prepared from amide S12 and 4-(4-piperidinyl)pyridine using general procedure B to furnish S43 in a 26% yield.

### EXAMPLE 2-TREATMENT OF P. AERUGINOSA

Various compounds of formula I in Example 1 above were tested to determine IC₅₀ of the compounds in conjunction with colistin relative to P. aeruginosa. IC₅₀ testing of the compounds was conducted to the following protocol. Overnight cultures of P. aeruginosa PA14 were back diluted into fresh LB to an OD600 of 2.5×10⁻³. Using polypropylene 96 well plates (Corning 3879), the potential antibiotic adjuvant was assayed with a 3-fold dilution starting with a 250 concentration. The concentration of colistin was held constant at sublethal concentration (0.25 to 0.375 pg/mL), using a freshly prepared colistin stock and minimizing transfers of colistin-containing solutions. After 17 h of aerobic growth at 37° C., 300 rpm, the absorbance at 600 nm was recorded.

Results of the IC₅₀ testing are provided in Table II

TABLE II
Compound IC₅₀ (μM)

| Compound | IC₅₀ |
|---|---|
| S17 | 14 |
| S20 | 15.5 |
| S22 | 15.5 |
| S24 | 14.4 |
| S26 | >250 |
| S27 | 2.6 |
| S28 | 0.8 |
| S29 | 1.5 |
| S30 | 2.6 |
| S32 | 57 |
| S34 | 88 |
| S38 | 2.6 |
| S39 | 15 |
| S40 | 50 |
| S41 | 95 |
| S42 | 70 |
| S43 | 75 |

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A compound of formula I: 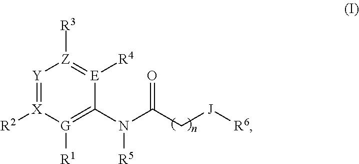
wherein E, G, X, Y, and Z are independently selected from C and N; and
wherein J is selected from the group consisting of O, S, CH₂, and NR⁷, wherein R⁷ is selected from the group consisting of hydrogen and alkyl; and
wherein R¹-R⁴ are independently selected from the group consisting of hydrogen, alkyl, fluoroalkyl, halo, and —OR⁸ wherein R⁸ is selected from the group consisting of hydrogen, alkyl and alkenyl; and
wherein R⁵ is selected from the group consisting of hydrogen and alkyl; and
wherein R⁶ is selected from the group consisting of hydrogen, alkenyl, alkynl, cycloalkyl, heterocycloalkyl, fused aryl, heteroaryl, fused heteroaryl, -arylene-alkynyl, -heteroarylene-alkynl, -arylene-aryl, -arylene-heteroaryl, -aryelene-thioalkyl, -arylene-(t-butyl), -heteroarylene-(t-butyl), -aryelene-amine, -heteroarylene-amine, -arylene-C(O)R⁹ wherein R⁹ is selected from the group consisting of hydrogen, alkyl, alkoxy, amine, and —OBn; and
wherein the cycloalkyl and heterocycloalkyl are optionally substituted with one or more substituents selected from the group consisting of alkyl, aryl, amine, heteroaryl, halo, hydroxy, and alkoxy; and
wherein n is an integer from 1 to 10.

2. The compound of claim 1, wherein J is O and R⁶ is selected from the group consisting of fused aryl, heteroaryl and fused heteroaryl.

3. The compound of claim 1, wherein J is O and R⁶ is selected from the group consisting of -aryelene-thioalkyl, -arylene-(t-butyl), -heteroarylene-(t-butyl), -arylene-alkynyl, and -heteroarylene-alkynl.

4. The compound of claim 1, wherein J is O and R⁶ is selected from the group consisting of -arylene-aryl and -arylene-heteroaryl.

5. The compound of claim 1, wherein J is O and R⁶ is -aryelene-C(O)R⁹ wherein R⁹ is selected from the group consisting of hydrogen, alkyl, alkoxy, amine, and —OBn.

6. The compound of claim 1, wherein J is O and R⁶ is selected from the group consisting of cycloalkyl and heterocycloalkyl.

7. A treatment for a gram negative bacterial infection comprising a cationic antimicrobial protein (CAP) in conjunction with a compound of formula I: 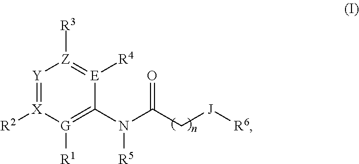
wherein E, G, X, Y, and Z are independently selected from C and N; and
wherein J is selected from the group consisting of O, S, CH₂, and NR⁷, wherein R⁷ is selected from the group consisting of hydrogen and alkyl; and
wherein R¹-R⁴ are independently selected from the group consisting of hydrogen, alkyl, fluoroalkyl, halo, and —OR⁸ wherein R⁸ is selected from the group consisting of hydrogen, alkyl and alkenyl; and
wherein R⁵ is selected from the group consisting of hydrogen and alkyl; and
wherein R⁶ is selected from the group consisting of hydrogen, alkenyl, alkynl, cycloalkyl, heterocycloalkyl, fused aryl, heteroaryl, fused heteroaryl, -arylene-alkynyl, -heteroarylene-alkynl, -arylene-aryl, -arylene-heteroaryl, -aryelene-thioalkyl, -arylene-(t-butyl), -heteroarylene-(t-butyl), -aryelene-amine, -heteroarylene-amine, -arylene-C(O)R⁹ wherein R⁹ is selected from the group consisting of hydrogen, alkyl, alkoxy, amine, and —OBn; and
wherein the cycloalkyl and heterocycloalkyl are optionally substituted with one or more substituents selected from the group consisting of alkyl, aryl, amine, heteroaryl, halo, hydroxy, and alkoxy; and
wherein n is an integer from 1 to 10.

8. The treatment of claim 7, wherein the CAP is colistin.

9. The treatment of claim 8, wherein the compound of formula (I) exhibits an IC₅₀ for inhibiting bacterial growth of less than 20 μM.

10. The treatment of claim 8, wherein the compound of formula (I) exhibits an IC₅₀ for inhibiting bacterial growth of less than 5 μM.

11. The treatment of claim 9, wherein the bacterial growth is growth of gram negative bacteria.

12. The treatment of claim 11, wherein the gram negative bacteria comprises P. aeruginosa.

13. The treatment of claim 7, wherein the compound of formula (I) and the CAP form a single composition.

14. The treatment of claim 7, wherein the compound of formula (I) and the CAP are separate compositions.

15. The treatment of claim 7, wherein J is O and R⁶ is selected from the group consisting of fused aryl, heteroaryl and fused heteroaryl.

16. The treatment of claim 7, wherein J is O and R⁶ is selected from the group consisting of -aryelene-thioalkyl, -arylene-(t-butyl), -heteroarylene-(t-butyl), -arylene-alkynyl, and -heteroarylene-alkynl.

17. The treatment of claim 7, wherein J is O and R⁶ is selected from the group consisting of -arylene-aryl and -arylene-heteroaryl.

18. The treatment of claim 7, wherein J is O and R⁶ is -aryelene-C(O)R⁹ wherein R⁹ is selected from the group consisting of hydrogen, alkyl, alkoxy, amine, and —OBn.

19. The treatment of claim 7, wherein J is O and R⁶ is selected from the group consisting of cycloalkyl and heterocycloalkyl.

20-28. (canceled)
